(19) United States
(12) Patent Application Publication
(10) Pub. No.: US 2010/0222675 A1
(43) Pub. Date: Sep. 2, 2010

(54) DEVICE AND METHOD FOR DETERMINING THE STATE OF ANCHORING OF AN IMPLANTED ENDOPROSTHESIS

(76) Inventor: Dietmar Ruwisch, Berlin (DE)

Correspondence Address: NOTARO, MICHALOS & ZACCARIA P.C., 100 DUTCH HILL ROAD, ORANGEBURG, NY, 10962, US

(21) Appl. No.: 12/682,111

(22) PCT Filed: Oct. 8, 2008
(86) PCT No.: PCT/EP08/63473

§ 371 Date: May 19, 2010

(30) Foreign Application Priority Data
Oct. 10, 2007 (DE) 10 2007 048 595.8

(51) Int. Cl. A61B 8/00

(52) U.S. Cl. 600/437

(57) ABSTRACT

In a device (10, 10′) for determining the state of anchoring of an implanted endoprosthesis (12, 12′), comprising means for oscillation of the endoprosthesis (12, 12′) and means for detecting the state of oscillation of the endoprosthesis (12, 12′), it is proposed that the means for oscillation of the endoprosthesis (12, 12′) are designed to emit a modulated ultrasound signal, comprising an ultrasound carrier signal and a tunable modulation signal.

This invention concerns a device for determining the state of anchoring of an implanted endoprosthesis, comprising:
means of stimulating the endoprosthesis to vibrate, and
means of capturing the vibration state of the endoprosthesis.

Such a device is known for the specific case of an implanted hip prosthesis from the article by R. Puers et al. “A telemetry system for the detection of hip prosthesis loosening by vibration analysis”, EUROSENSORS XIII, 13th European Conference on Solid-State Transducers, 12th-15 Sep. 1999, Den Haag, pp. 757-760. In this case the means of stimulating the endoprosthesis to vibrate include a jolting device, which is placed on the tissue in the region of the thighbone of a patient. The jolting device causes the whole thigh, and thus also the thighbone and the hip prosthesis implanted in it, to vibrate. As the means of capturing the vibration state, this device of the prior art includes an acceleration sensor which is built into the upper region of the hip prosthesis, and depending on acceleration or deceleration generates an appropriate signal. In particular by jerky stresses on the acceleration sensor, a distinctive signal is triggered. It has been shown that the signals of the acceleration sensor depend to a large extent on whether the hip prosthesis is firmly anchored in the bone, resulting in direct transmission of the forces acting on the bone to the sensor, or whether the prosthesis is loosened, which interferes with the direct force transmission. The latter case is expressed in the acceleration behaviour of the prosthesis, and accordingly in the output signal of the acceleration sensor.

This device of the prior art has various disadvantages. On the one hand, causing the thigh to vibrate is relatively unpleasant for the patient, especially because, in view of the damping of the vibrations in the tissue, large vibration amplitudes are necessary for obtaining measurement signals which can be evaluated. On the other hand, in the case of this device the stimulation of the prosthesis vibration is relatively badly defined, since for example it depends on the precise position where the jolting device is placed, as well as on the patient's build, the thickness of various tissue and fat layers, etc.

Additionally, with this device not only the prosthesis which is actually of interest, but the whole bone system in which it is implanted, is caused to vibrate. In practice, therefore, it has been shown that with such a system, precisely defined stimulation of vibration of the prosthesis can be implemented only with difficulty.

It is therefore the object of this invention to develop a generic device further, so that with less stress on the patient, it is made possible to determine the anchoring state of the implanted endoprosthesis in a way which is more precise and can be better reproduced.

According to the invention, this object is achieved by the means of stimulating the endoprosthesis to vibrate being designed to emit a modulated ultrasound signal, comprising an ultrasound carrier signal and a tunable modulation signal.

The frequency of the modulation signal is tunable so that at the interface between the endoprosthesis and the surrounding tissue, in particular the surrounding bone, it causes a controllable energy transfer to the implanted endoprosthesis. This puts the endoprosthesis into forced vibration at the frequency of the modulation signal. The purpose of the ultrasound carrier signal is essentially “only” to transport the modulation signal through the human or animal body to the endoprosthesis. In this way, using the modulation signal of tunable frequency and its energy transfer to the endoprosthesis, stimulation of vibration of the prosthesis can be achieved without simultaneous direct stimulation of vibration of the surrounding bone. Use of ultrasound signals is known from numerous medical investigation procedures, e.g. imaging procedures, and for the patient is not usually associated with stress or even pain.

The modulation signal which is overlaid over the ultrasound carrier signal is intended to ensure that at the interface between endoprosthesis and surrounding bone, an energy transfer to the prosthesis occurs, and itself stimulates forced vibration. In principle, for this purpose, frequency modulation of the carrier signal using the modulation signal would be considered. However, it is preferred that the modulated ultrasound signal is an amplitude-modulated ultrasound signal, which technically is specially easy to generate.

The function of the ultrasound carrier signal is essentially to transport the modulation signal to the interface between the endoprosthesis and the surrounding bone. The frequency of the ultrasound carrier signal is therefore preferably chosen so that the material of a body in which the endoprosthesis is implanted is penetrated essentially without interference. For example, various layers of skin, layers of fat, bones etc. should be seen as “material” of the body.

In the usual case of an endoprosthesis which is implanted in a human or animal body, the usual result of this is that the frequency of the ultrasound carrier signal is within a frequency interval of 20 kHz to 40 MHz, and preferably approximately 100 kHz.

To determine the anchoring state of the implanted endoprosthesis, in principle it would be possible to set a predetermined frequency of the modulation signal, consequently to stimulate the prosthesis to forced vibration at just this frequency, and for example to investigate the amplitudes of the forced vibration using the means of capturing the vibration state. However, preferably it is provided that the means of stimulating vibration are designed to tune the frequency of the modulation signal in a frequency interval which includes at least one expected resonant frequency of the endoprosthesis. The means of stimulating vibration then make it possible to find, as the frequency of the forced vibration, a natural frequency of the implanted endoprosthesis, so that the latter is stimulated to resonant vibration. Because of the energy transfer, which is maximal in this case, from the irradiated total ultrasound signal to the implanted prosthesis, the means of capturing the vibration state of the prosthesis can then supply specially clear signals, which make it possible to determine whether the endoprosthesis has become loose, in particular using a comparison of a currently found resonant frequency of the endoprosthesis with a resonant frequency which was established in an earlier investigation.

Usefully, therefore, the frequency interval for tuning the modulation signal frequency should be between 100 Hz and 10 kHz. It has been shown that the (often multiple) resonant frequencies of a loose prosthesis (e.g. natural frequencies of bending vibrations or torsion vibrations in various spatial directions) are regularly in this frequency interval.

In a simple embodiment of the invention, it is provided that the means of capturing the vibration state of the endoprosthesis include a sensor which is attached to the endoprosthesis, and which is designed to capture the vibration state of the endoprosthesis, and a transponder unit, which is designed to transmit vibration measurement signals output by the sensor to a signal processing unit, the sensor being, for example, an acceleration, vibration and/or position measurement sensor and/or a laser vibrometer. Use of such acceleration or related sensors to capture the vibration state of an implanted endoprosthesis is generally known from the prior art. Reference can be made again to the article by R. Puers et al., which was mentioned in the introduction, and for example to DE 10342823A1, to which in this respect reference is made in full.

Thus in this embodiment, the device according to the invention always makes it possible to determine the anchoring state of the implanted endoprosthesis, if the latter is equipped with an acceleration or similar sensor which is known per se from the prior art.

In a further development of the invention, an embodiment which makes it possible to determine the anchoring state independently of the existence of such a sensor, either because the sensor is no longer functional or because the prosthesis was originally implanted without such a sensor, is proposed. In this further embodiment, it is provided that the means of capturing the vibration state of the endoprosthesis include an ultrasound receiver and an evaluation unit. The ultrasound receiver and the evaluation unit which is connected to it then determine the anchoring state of the prosthesis on the basis of the ultrasound signals which the latter emits at each forced vibration. In particular, the evaluation unit is usefully designed to analyse ultrasound signals which are reflected by the endoprosthesis and received by the ultrasound receiver. This embodiment thus makes it possible, using the modulated ultrasound signal, to stimulate the implanted endoprosthesis in resonance, after the frequency of the tunable modulation signal has been set to the natural frequency of the prosthesis. It has been shown that a prosthesis which has thus been stimulated to forced vibration causes a frequency and/or phase modulation of the reflected ultrasound signal compared with the irradiated ultrasound. Thus in this embodiment of the invention too, the resonance case can be detected by tuning the frequency of the modulation of the irradiated ultrasound signal until the modulation effects (frequency and/or phase modulation) which are observed in the reflected ultrasound signal using the ultrasound receiver and the evaluation unit which is associated with it are maximal.

To simplify the signal evaluation, it is usefully provided that the means of stimulating the endoprosthesis to vibrate are designed to switch off the modulation signal and emit the ultrasound carrier signal with no modulation signal. If the prosthesis has been stimulated by the modulation signal far from resonance, the forced vibration dies away extremely quickly. Then, after the modulation signal is switched off, it is hardly possible to demonstrate modulation effects in the reflected ultrasound signal. However, if the resonance case has occurred, i.e. the modulation signal, because of a suitable choice of frequency, has stimulated a natural frequency of the implanted and loosened endoprosthesis, the latter vibrates for a relatively long time even after the modulation signal is switched off, so that modulation effects can be observed in the reflected ultrasound signal, in particular in the form of frequency modulation.

Thus in all variants of this embodiment, which is based on an ultrasound receiver and a connected evaluation unit, it is provided that the analysis includes a frequency analysis. Also, investigation of events in a patient's body using frequency analysis of reflected ultrasound signals is generally known in the field of imaging procedures, in particular in the form of Doppler analysis as a specially simple form of frequency analysis (cf. EP 1769747A1, for example).

Usefully, the means of stimulating the endoprosthesis to vibrate and the ultrasound receiver can comprise a common ultrasound transmission/reception unit. Such combined ultrasound transmitters/receivers are also known, both in the field of imaging ultrasound procedures and, for example, in the field of lithotripsy.

In the case of the preferred embodiments and variants described above, the device according to the invention is used to capture the resonant frequency of the implanted endoprosthesis in the context of an investigation of the patient, and to compare it with a resonant frequency which was determined in an earlier investigation. Changes of the resonant frequency indicate that the anchoring state of the prosthesis has changed, which usually leads to the conclusion that it has become loose. On the other hand, if it is established that the determined resonant frequency essentially corresponds to that of an earlier investigation, to this extent at least there is no indication of loosening of the prosthesis. An inspection operation, which might be carried out otherwise, can be omitted in this case. Usefully, the device according to the invention should therefore be in such a form that the means of capturing the vibration state of the endoprosthesis include a memory unit for storing earlier measurement results, in particular previously established resonant frequencies of the endoprosthesis. In particular, in the embodiment described above, in which a sensor attached to the endoprosthesis is used, the memory unit can be associated with this sensor and also attached to the prosthesis, so that the patient virtually carries his or her measurement results with him or her.

In the further embodiment of the invention described above, which works without such a sensor, the means of capturing the vibration state of the endoprosthesis, and thus also the above-mentioned memory unit, are outside the patient, e.g. as part of the evaluation unit or of a computer which controls all components of the device according to the invention.

The comparison of a currently determined resonant frequency of the endoprosthesis with a previously determined resonant frequency can be carried out by appropriately trained medical or technical personnel. However, usefully it can also be provided that the means of capturing the vibration state of the endoprosthesis include a comparison unit for automatic comparison of current and previous measurement results.

The invention also concerns a method of determining the anchoring state of an implanted endoprosthesis using a device according to the invention, the means of stimulating the endoprosthesis to vibrate emitting the modulated ultrasound signal in the direction of the endoprosthesis, and the vibration state of the endoprosthesis being captured by the means of capturing the vibration state of the endoprosthesis.

Preferred embodiments of the invention are explained below, purely as examples and without any restriction, on the basis of the attached drawings, of which:

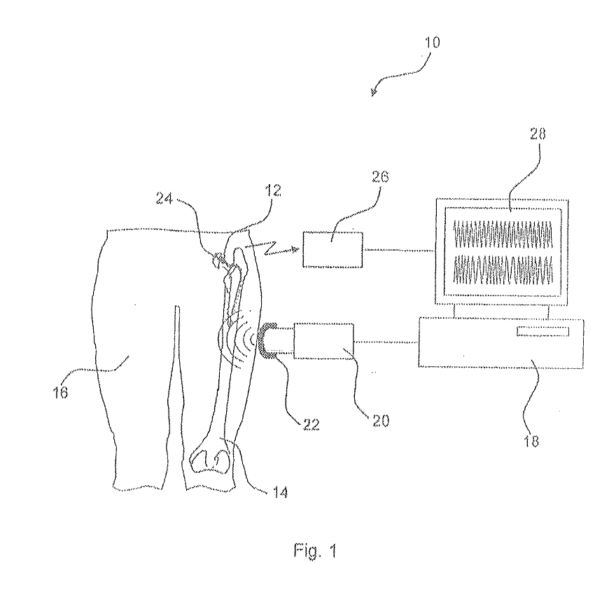

FIG. 1 shows a schematic overall view of a first embodiment of the device according to the invention;

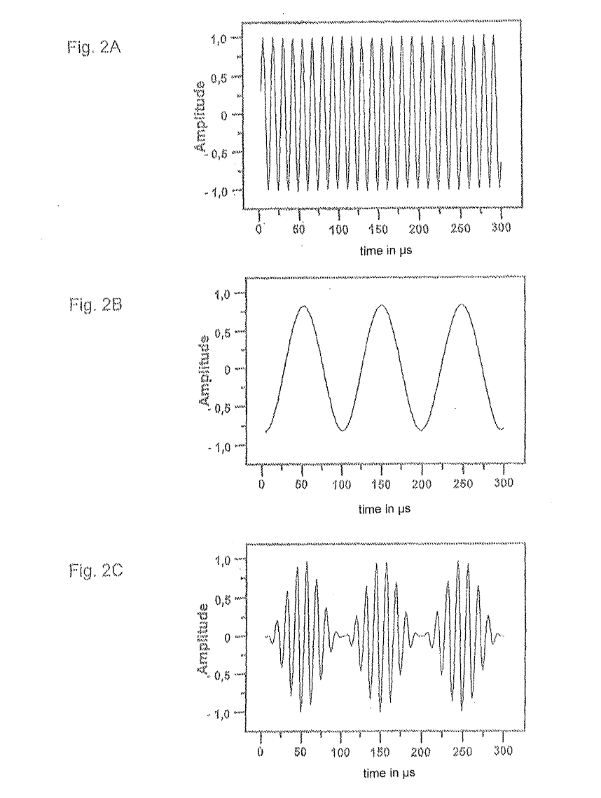

FIGS. 2A, 2B and 2C show typical courses over time of an ultrasound carrier signal, a modulation signal and the resulting amplitude-modulated ultrasound signal;

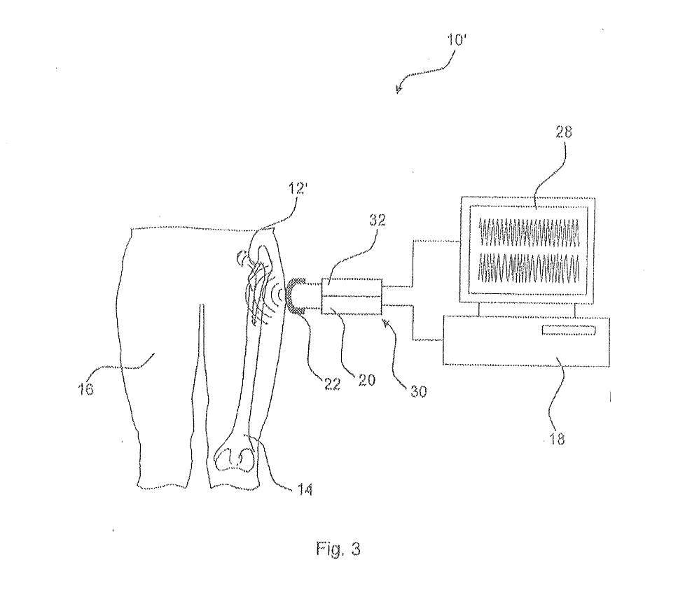

FIG. 3 shows a schematic overall view, similar to FIG. 1, of a second embodiment of the device according to the invention; and

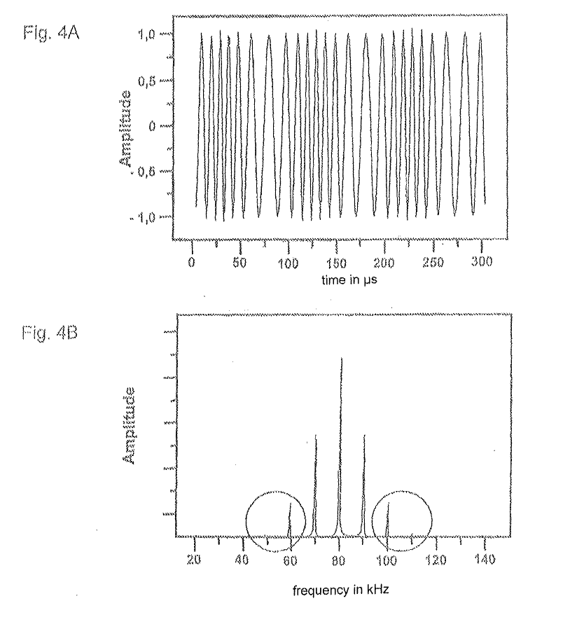

FIGS. 4A and 4B show a course over time and a frequency spectrum of an ultrasound signal which is received using the device from FIG. 3.

FIG. 1 shows a schematic view of a first embodiment of the device 10 according to the invention to determine the anchoring state of an implanted endoprosthesis 12. With no restriction, in FIG. 1 the case of a hip prosthesis 12, which is implanted in the thighbone 14 of a patient 16, is shown schematically. It should be pointed out here that the device according to the invention, in all embodiments, can of course be used with other endoprostheses, e.g. artificial knee joints.

The device 10 according to the invention is intended to make it possible to determine the anchoring state of the prosthesis 12 in the thighbone 14, and thus, if appropriate, to make an inspection operation, which would otherwise classically have been carried out for this purpose, superfluous. According to the invention, for this purpose the first embodiment of the device 10, shown in FIG. 1, includes an ultrasound emission unit 20 which is controlled by a central control computer 18. The ultrasound emission unit 20 includes, at its left-hand end in FIG. 1, a coupling cushion 22, such as is known in principle from ultrasound devices which are used in the medical sector. To investigate the patient 16, the ultrasound emission unit 20, with its coupling cushion 22, is placed in contact with the thigh of the patient 16, and ultrasound waves are emitted in the direction of the prosthesis 12, as symbolised in FIG. 1 by schematically drawn wavy lines.

On the basis of control by the control computer 18, the ultrasound emission unit 20, using the coupling cushion 22, emits an amplitude-modulated ultrasound signal, which is based on an ultrasound carrier signal which is shown as an example in FIG. 2A, and which is modulated by a tunable modulation signal which is shown as an example in FIG. 2B. The modulated total ultrasound signal resulting from this modulation is shown in FIG. 2C. In the case of the ultrasound waves which are shown as examples in FIGS. 2A to 2C, the frequency of the ultrasound carrier signal is approximately 80 kHz, and the frequency of the modulation signal is approximately 10 kHz.

The modulated total ultrasound signal shown in FIG. 2C passes through the tissue of the patient 16, from the contact region of the coupling cushion 22 to the internal interface between the thighbone 14 and the prosthesis 12, essentially without loss. At this interface, the modulated ultrasound signal shown in FIG. 2C puts the prosthesis 12 into forced vibration at the frequency of the modulation signal which is shown schematically in FIG. 2B.

This forced vibration of the prosthesis 12, as is known in principle for example from DE 10342823A1 for a different type of vibration stimulation, is captured using a sensor 24, which in the embodiment of FIG. 1, for example, is housed in the head of the prosthesis 12. Via a transponder unit, which is built into the sensor 24, corresponding vibration measurement signals, in particular information about the amplitude and frequency of the forced vibration of the prosthesis 12, are transmitted by radio to a signal processing unit 26, which in turn is connected to the central control computer 18. It is of course understood that the signal processing unit 26 can also be in the form of an integrated part of the computer 18.

The computer 18 controls the ultrasound emission unit 20 so that the frequency of the modulation signal is tuned in a frequency interval of typically about 100 Hz to about 10 kHz. As explained above, the prosthesis 12 is stimulated to forced vibration at the currently set modulation frequency. Thus whenever the modulation frequency reaches one of usually multiple natural frequencies of the implanted prosthesis 12, e.g. a natural frequency of bending vibration or torsion vibration, a resonance case occurs, i.e. the prosthesis 12 vibrates at specially strongly pronounced vibration amplitudes, and this vibration also noticeably continues after the modulation is switched off.

The control computer 18 is designed to investigate the vibration measurement signals which are supplied to it via the sensor 24 and signal processing unit 26 automatically for the occurrence of resonances, in particular to identify and store resonant frequencies. If it is established that the resonant frequencies which occur during an investigation of the patient 16 are essentially identical to the resonant frequencies which were observed in a past investigation, to that extent there is no indication of a loosening of the prosthesis 12, the vibration behaviour of which has evidently not changed. On the other hand, if a displacement of at least one resonant frequency in comparison with one of the earlier investigations is observed, this represents a strong indication that at least one of the possible natural vibrations of the prosthesis 12 has changed, indicating a loosening of the prosthesis 12.

As indicated schematically in FIG. 1, the central control computer 18 includes a screen 28, on which, for example, the radiated ultrasound waves can be shown. Usefully, the control computer 18 is also designed to display, in the course of the investigation of the patient 16, the currently determined resonant frequencies, as well as, for example, appropriate notification if a change compared with stored earlier measurement results is established. For this purpose, the control computer 18 is usefully equipped with a memory unit (not shown in the figures) to store the measurement results, in particular previously established resonant frequencies of the prosthesis 12, and advantageously also with a comparison unit for automatic comparison of current measurement results with earlier measurement results.

Whereas the first embodiment of the device 10 according to the invention, shown schematically in FIG. 1, can fall back on known technologies from the prior art with respect to the means of capturing the vibration state of the prosthesis 12, in the form of the sensor 24 and signal processing unit 26, below, on the basis of FIGS. 3, 4A and 4B, a second embodiment of the device 10′ according to the invention, which can be used even with prostheses 12′ which have no such sensor, is presented. In the second embodiment of the device 10′ according to the invention, shown schematically in FIG. 3, the central control computer 18 controls a combined ultrasound transmission/reception unit 30. This comprises an ultrasound transmission unit 20, which is similar to that of the first embodiment from FIG. 1, an ultrasound reception unit 32, and a coupling cushion 22, which is assigned to both units 20, 32. On the transmission side, i.e. with respect to the transmission of ultrasound waves using the transmission unit 20 and coupling cushion 22 in the direction of the implanted prosthesis 12′, reference can be made to the first embodiment of FIG. 1. In particular, the emitted ultrasound carrier signal, the tunable modulation signal and the modulated ultrasound signal which results from them again correspond to the wave courses which are shown in FIGS. 2A, 2B and 2C respectively.

However, the second embodiment of the device 10′ according to the invention differs from the first embodiment on the reception side, i.e. with respect to the means of capturing the vibration state of the prosthesis 12′. For this purpose the ultrasound reception unit 32 and coupling cushion 22 act as an ultrasound receiver, which receives ultrasound signals which are reflected by the prosthesis 12′ and feeds them to an evaluation unit in the form of part of the control computer 18. This is explained below on the basis of FIGS. 2A to 2C, 4A and 4B.

First, the control computer 18 again controls the ultrasound transmission unit 20 so that it emits an amplitude-modulated total ultrasound signal, corresponding to the one in FIG. 2C, in the direction of the prosthesis 12′. The modulation frequency is again tuned by the control computer 18. Now, in the second embodiment of the device 10′ according to the invention, the ultrasound signals reflected by the vibrating prosthesis 12′ are measured using the ultrasound reception unit 32, preferably after switching off the modulation signal, as follows.

When the modulated ultrasound signal is emitted according to FIG. 2C, the prosthesis 12′ in the thighbone 14 is stimulated to forced vibration, with the result that the ultrasound signal reflected by the prosthesis 12′ has a frequency shift, similarly to the case of the known (ultrasound) Doppler effect. In particular, in the ultrasound signal reflected by the vibrating interface of the prosthesis 12′, there are frequency components which correspond to the typical line spectrum of a frequency or phase modulation. In particular, secondary lines occur at the positive and negative integer multiples of the modulation frequency. These secondary lines are specially pronounced in the resonance case, where the analysis must concentrate on 2nd and 3rd order secondary lines (i.e. at the carrier frequency plus or minus twice and three times the modulation frequency), since the amplitude modulation of the irradiated ultrasound signal already results in pronounced 1st order secondary lines (i.e. at the carrier frequency plus or minus the modulation frequency), but not in higher order secondary lines. To observe the modulation effects in the ultrasound signal reflected by the prosthesis 12′, the control computer 18 is usefully designed to switch off the transmission-side amplitude modulation at regular time intervals, so that temporarily “only” the ultrasound carrier signal continues to be irradiated. In this way, even the 1st order secondary lines can be used for evaluation. The reflected ultrasound signal which is observed in the case of resonance immediately after the modulation is switched off is shown in FIG. 4A. The occurrence of vibration components of higher and lower frequency than the underlying carrier frequency, corresponding to the above-mentioned addition and subtraction of the vibration frequency of the stimulated prosthesis 12′, is clearly seen.

FIG. 4A shows a corresponding wave representation of the reflected ultrasound signal after the transmission-side modulation is switched off, for the case that a resonance case has been achieved using the modulation. Far from the resonance, hardly noticeable frequency changes in the reflected ultrasound signal can be observed, and such frequency modulations outside resonance die away significantly faster than in the resonance case.

The control computer 18 is designed to carry out a frequency analysis of the received ultrasound waves which are shown schematically in FIG. 4A, in a way which is known per se. The result of such a frequency analysis is shown in FIG. 4B. What is seen first here is a central line corresponding to the ultrasound carrier frequency, in this case approximately 80 kHz. There are also 1st order secondary lines at approximately 80±10 kHz, i.e. corresponding to the sum and difference of the ultrasound carrier frequency and the momentarily irradiated modulation frequency of the amplitude modulation corresponding to FIG. 2B.

The resonance case can now be detected on the basis of the occurrence of further secondary lines, which are circled in FIG. 4B. Far from the resonance, i.e. if the irradiated modulation frequency does not correspond to a natural frequency of the possibly loosened prosthesis 12′, it is not or hardly possible to observe these secondary lines, since they correspond to the characteristic line spectrum of a frequency or phase modulation which is generated on the reflected carrier signal by the vibration of the prosthesis, whereas the amplitude modulation of the irradiated wave results only in 1st order secondary lines. 1st order secondary lines also belong to the spectrum of a frequency or phase modulation, but are covered by the amplitude modulation of the irradiated wave unless the amplitude modulation is switched off.

Thus, using frequency analysis of the received ultrasound signal, it is also possible to determine reliably every resonant frequency of the prosthesis 12′, and to compare them with corresponding measurement results of earlier investigations, to discover any loosening of the prosthesis.

The device according to the invention is of course not restricted to the embodiments which are presented purely as examples. Thus, as explained above, the prosthesis 12, 12′ is not necessarily a hip prosthesis, but can be any other kind of endoprosthesis. It is understood that in this case, different frequency intervals for the ultrasound signals which are used come into consideration, and in particular that the frequency of the tunable modulation signal must be adapted to the vibration conditions, which are changed compared with a hip prosthesis. The embodiments which are presented on the basis of FIGS. 1 and 3 can of course also be combined with each other, i.e. even a prosthesis 12 which is equipped with a sensor 24 can in principle be investigated for loosening according to the second embodiment of FIG. 3, e.g. to check the results of an investigation based on the sensor 24.

The above-mentioned memory unit for storing earlier measurement results, in particular previously established resonant frequencies of the prosthesis 12, 12′, can first be provided as an integrated part of the control computer 18. However, if a prosthesis 12 with a built-in sensor 24 is used, the memory unit can also be provided as part of the sensor 24. In this case, the patient 16 virtually carries the results of earlier investigations with him or her.

Additionally, it is understood that the memory unit can also be in the form of an external memory medium, e.g. in the form of a patient card of the patient 16.

1. Device (10, 10′) for determining the state of anchoring of an implanted endoprosthesis (12, 12′), comprising:
means of stimulating the endoprosthesis (12, 12′) to vibrate, and
means of capturing the vibration state of the endoprosthesis (12, 12′), characterized in that the means of stimulating the endoprosthesis (12, 12′) to vibrate are designed to emit a modulated ultrasound signal, comprising an ultrasound carrier signal and a tunable modulation signal.

2. Device (10, 10′) according to claim 1, characterized in that the modulated ultrasound signal is an amplitude-modulated ultrasound signal.

3. Device (10, 10′) according to claim 1, characterized in that the frequency of the ultrasound carrier signal is chosen so that the material of a body in which the endoprosthesis (12, 12′) is implanted is penetrated essentially without interference.

4. Device (10, 10′) according to claim 3, characterized in that the frequency of the ultrasound carrier signal is within a frequency interval of 20 kHz to 40 MHz, and preferably approximately 100 kHz.

5. Device (10, 10′) according to claim 1, characterized in that the means of stimulating vibration are designed to tune the frequency of the modulation signal in a frequency interval which includes at least one expected resonant frequency of the endoprosthesis (12, 12′).

6. Device (10, 10′) according to claim 5, characterized in that the frequency interval for tuning the modulation signal frequency is between 100 Hz and 10 kHz.

7. Device (10, 10′) according to claim 1, characterized in that the means of capturing the vibration state of the endoprosthesis (12, 12′) include a sensor (24) which is attached to the endoprosthesis (12, 12′), and which is designed to capture the vibration state of the endoprosthesis (12, 12′), and a transponder unit, which is designed to transmit vibration measurement signals output by the sensor (24) to a signal processing unit (26).

8. Device (10, 10′) according to claim 7, characterized in that the sensor (24) is an acceleration, vibration and/or position measurement sensor and/or a laser vibrometer.

9. Device (10, 10′) according to claim 1, characterized in that the means of capturing the vibration state of the endoprosthesis (12, 12′) include an ultrasound receiver and an evaluation unit.

10. Device (10, 10′) according to claim 9, characterized in that the evaluation unit is designed to analyse ultrasound signals which are reflected by the endoprosthesis (12, 12′) and received by the ultrasound receiver.

11. Device (10, 10′) according to claim 9, characterized in that the means of stimulating the endoprosthesis (12, 12′) to vibrate are designed to switch off the modulation signal and emit the ultrasound carrier signal with no modulation signal.

12. Device (10, 10′) according to claim 10, characterized in that the analysis includes a frequency analysis.

13. Device (10, 10′) according to claim 9, characterized in that the means of stimulating the endoprosthesis (12, 12′) to vibrate and the ultrasound receiver comprise a common ultrasound transmission/reception unit (30).

14. Device (10, 10′) according to claim 1, characterized in that the means of capturing the vibration state of the endoprosthesis (12, 12′) include a memory unit for storing earlier measurement results, in particular previously established resonant frequencies of the endoprosthesis (12, 12′).

15. Device (10, 10′) according to claim 14, characterized in that the means of capturing the vibration state of the endoprosthesis (12, 12′) include a comparison unit for automatic comparison of current and previous measurement results.

16. Method of determining the anchoring state of an implanted endoprosthesis (12, 12′) according to claim 1, characterized in that the means of stimulating the endoprosthesis (12, 12′) to vibrate emit the modulated ultrasound signal in the direction of the endoprosthesis (12, 12′), and that the vibration state of the endoprosthesis (12, 12′) is captured by the means of capturing the vibration state of the endoprosthesis (12, 12′).
